(19) United States
(12) Patent Application Publication
(10) Pub. No.: US 2023/0263426 A1
(43) Pub. Date: Aug. 24, 2023

(54) SPIROMETRY METHODS TO DIAGNOSE MILD AND EARLY AIRFLOW OBSTRUCTION

(71) Applicant: THE UAB RESEARCH FOUNDATION, Birmingham, AL (US)

(72) Inventors: Surya P. Bhatt, Birmingham, AL (US); Arie Nakhmani, Birmingham, AL (US); Sandeep Bodduluri, Birmingham, AL (US)

(73) Assignee: THE UAB RESEARCH FOUNDATION, Birmingham, AL (US)

(21) Appl. No.: 18/041,229

(22) PCT Filed: Aug. 13, 2021
(86) PCT No.: PCT/US2021/045868

§ 371 Date: Feb. 10, 2023

Related U.S. Application Data
(60) Provisional application No. 63/065,165, filed on Aug. 13, 2020.

(51) Int. Cl. A61B 5/087; A61B 5/091; G16H 50/20; G16H 20/00

(52) U.S. Cl. CPC A61B 5/087; A61B 5/091; G16H 50/20; G16H 20/00

(57) ABSTRACT

The present disclosure relates to a method of detecting airflow obstruction using one or more novel metrics associated with a spirometry reading for a subject. The method includes obtaining spirometry data from a subject, generating a first measurement curve and a second measurement curve based on the obtained data for the subject, performing at least a first curve-fitting on the first measurement curve using a Least Absolute Residuals algorithm to estimate a function which closely approximates the spirometry data for the subject by minimizing a sum of absolute deviation, determining a first metric that describes a rate of volume increase based on the estimated function, comparing the first metric to one or more threshold values, and determining a presence or absence of airflow obstruction for the subject based on the comparison of the first metric to the one or more threshold values.

### CROSS-REFERENCE TO RELATED APPLICATION

The present application claims the benefit and priority of U.S. Provisional Patent Application No. 63/065,165, filed on Aug. 13, 2020, which is hereby incorporated by reference in its entirety for all purposes.

### FIELD

The present disclosure relates to spirometry techniques to diagnose mild and early airflow obstruction for a subject.

### BACKGROUND

There are approximately 14 million individuals who have been diagnosed to have chronic obstructive pulmonary disease (COPD) in the United States alone, and an additional 10 million individuals are suspected to have undiagnosed COPD. Diagnosing COPD relies on identifying a presence of airflow obstruction for a subject using spirometry.

Spirometry is a noninvasive technique that involves measuring air volumes inspired and expired by lungs using a spirometer device. Conventional spirometry measures include an low ratio of the forced expiratory volume in 1 second (FEV1) to a forced vital capacity (FVC) in order to identify airflow obstruction. However, these spirometry measures cannot accurately identify a presence of structural lung disease as evidenced on computed tomography (CT) imaging. Approximately 50% of individuals with risk factors for COPD may have spirometry measurements within a normal range (e.g., a FEV1/FVC ratio above a threshold value) based on traditional criteria and yet show evidence of structural lung disease in the form of emphysema and airway wall thickening based on CT imaging.

Conventional spirometry measures are also deficient when it comes to detecting mild disease or an early stage of disease. In particular, conventional techniques for detecting mild disease involve estimating an expiratory flow and/or examining the slope of some portion of a flow-volume curve and/or difference in angle of curvature during forced expiration. However, these techniques are limited by small sample sizes and a lack of validation against structural lung disease. Early detection and treatment of COPD is desired as it may improve quality of life and longevity of affected individuals. Conventional measures are further significantly influenced by age, height, and sex, which necessitates periodic updates of reference equations derived from a normal population.

Embodiments of the present disclosure aim to address these and other problems.

### SUMMARY

Disclosed are methods, systems, and computer readable storage media for diagnosing mild and early airflow obstruction for a subject. The methods, systems, and computer readable storage media may be embodied in a variety of ways.

In some embodiments, a method is provided that includes obtaining, using a spirometer, data corresponding to one or more expiratory air measurements for a subject; generating a first measurement curve and a second measurement curve based on the obtained data for the subject; performing at least a first curve-fitting on the first measurement curve, where the performing the first curve-fitting on the first measurement curve comprises: applying a first function to the first measurement curve to estimate a function which closely approximates the obtained data for the subject by minimizing a sum of absolute deviation, where the first function includes Least Absolute Residuals; and determining a first metric based on the estimated function, where the first metric describes a rate of volume increase; comparing the first metric to one or more threshold values; and determining a presence or absence of airflow obstruction for the subject based on the comparison of the first metric to the one or more threshold values.

In some embodiments, the method further includes: comparing the first metric to: (i) a first quartile that corresponds to a reference Parameter D value of a normal subject that is less than −5.077; (ii) a second quartile that corresponds to an average Parameter D value between −5.076 and −3.631 (iii) a third quartile that corresponds to an average Parameter D value between −3.630 to −2.209; and a fourth quartile that corresponds to an average Parameter D value equal to or greater than −2.209; and determining a cumulative rate of survival for the subject based on the comparison, wherein subjects within the fourth quartile have significantly higher mortality compared to subjects in the first-third quartile.

In some embodiments, the first measurement curve indicates a volume of air exhaled over a time period and the second measurement curve indicates a rate of flow over a volume of air exhaled.

In some embodiments, the method further includes: determining the presence of the airflow obstruction is associated with chronic obstructive pulmonary disease (COPD); and generating and implementing a treatment plan for COPD for the subject based on the determined presence of the airflow obstruction and/or the determined cumulative rate of survival for the subject.

In some embodiments, the method further includes: performing at least a second curve-fitting on the second measurement curve, wherein the performing the second curve-fitting on the second measurement curve comprises: applying a second function with at least two or more linear segments to a portion of the second measurement curve; and determining a second metric based on an intersection point for the at least two or more linear segments of the piece-wise function; performing at least a third curve-fitting on the second measurement curve, wherein the performing the third curve-fitting on the second measurement curve further comprises: applying a third function around a highest point of the second measurement curve using a least squares minimization; and determining a third metric from the highest point of the second measurement curve and a point where the applied function deviates from the second measurement curve; comparing the first metric, the second metric, and the third metric to a plurality of threshold values; and determining the presence or absence of airflow obstruction for the subject based on the comparison of the first metric, the second metric, and the third metric to the plurality of threshold values.

In some embodiments, a method is provided that includes determining, by a user, a diagnosis of a subject based on a result generated from data points on expiratory spirometry curves using part or all of one or more techniques disclosed herein and potentially selecting, recommending and/or administering a particular treatment to the subject based on the diagnosis.

In some embodiments, a method is provided that includes determining, by a user, a treatment to select, recommend and/or administer to a subject based on a result generated from data points on expiratory spirometry curves using part or all of one or more techniques disclosed herein.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows a model fitting of a volume-time curve and computation of Parameter D.

FIGS. 2A-2B show flow-volume curves illustrating: (2A) a computation of a Transition Point, and (2B) a computation of a breaking point and a change of volume from the maximum to the breaking point (Transition Distance).

FIG. 3 shows a table displaying associations between new spirometry metrics and CT disease and a respiratory morbidity. Adjustments were made for age, sex, race, and BMI as well as scanner type in the case of CT parameters.

FIGS. 4A-4B show plots associating quartiles of an updated Parameter D calculated using LAR with a respiratory morbidity.

FIG. 5 shows a table displaying a comparison of demographics, imaging and respiratory morbidity between concordant and discordant groups by Parameter D and a FEV1/FVC ratio <0.70.

FIG. 6 shows a table displaying odds ratios of COPD diagnostic criteria for predicting imaging measures of COPD. Adjustments were included for age, sex, race, and BMI for each subject and a scanner type. All comparisons made for each group in reference with normal controls.

FIG. 7 shows a lung CT image and a volume-flow curve for a 54 year old African American male subject with a 34 pack-year smoking history.

FIGS. 8A-8B shows a correlation plot (8A) associating Parameter D with subject age and a correlation plot (8B) associating Parameter D with subject height.

FIGS. 9A-9B shows a correlation plot (9A) associating a FEV1 value with subject age and a correlation plot (9B) associating a FEV1 value with subject height. Both plots additionally include a legend for identifying a gender distribution.

FIGS. 10A-10B shows a correlation plot (10A) associating Parameter D with subject age and a correlation plot (10B) associating Parameter D with subject height. Both plots additionally include a legend for identifying a gender distribution.

FIG. 11 shows a block diagram that illustrates a computing environment for obtaining and processing spirometry data in accordance with various embodiments.

FIG. 12 shows a flowchart illustrating a process for obtaining and processing spirometry data to determine a presence or absence of airflow obstruction for a subject in accordance with various embodiments.

### DETAILED DESCRIPTION

While certain embodiments are described, these embodiments are presented by way of example only, and are not intended to limit the scope of protection. The apparatuses, methods, and systems described herein may be embodied in a variety of other forms. Furthermore, various omissions, substitutions, and changes in the form of the example methods and systems described herein may be made without departing from the scope of protection.

### I. Overview

The present disclosure describes techniques for detecting mild airflow obstruction using novel spirometry metrics. Techniques involve using all data points on expiratory spirometry curves to assess their shape and improve detection of mild airflow obstruction and/or structural lung disease. Expiratory spirometry curves may include at least a volume-time curve illustrating a volume of air exhaled over a time period and a flow-volume curve illustrating a rate of air flow over a volume of air exhaled. In particular, techniques include analyzing one or more metrics of spirometric data: (i) a shape of the volume-time curve (e.g., referred to herein as “Parameter D”), (ii) a “Transition Point”, and/or (iii) a “Transition Distance” associated with the flow-volume curve.

The diagnosis of COPD relies on a demonstration of airflow obstruction. Airflow obstruction may be associated with one or more respiratory symptoms and/or structural lung disease. Traditional spirometry metrics are unable to correctly identify a number of subjects with respiratory symptoms or structural lung disease (e.g., indicated via CT imaging). In such instances, novel spirometry metrics can be derived from a flow-volume curve and a volume-time curve generated by a spirometer reading for a subject. In particular, the novel metrics are generated by modelling an entire flow-volume curve and a volume-time curve using a mathematical function using a function-fitting process. These novel metrics can then be used to identify airflow obstruction, particularly for subjects who are diagnosed as not having any respiratory disease by traditional criteria.

The method of the present disclosure identifies at least an additional 9.5% of subjects with COPD, including subjects with a mild disease or at an early stage of the disease, that are not detected using traditional spirometry measures. Novel metrics of the proposed method may be incorporated in a hardware spirometer device and/or as a post-processing software step for analyzing spirometry curves generated by a spirometer device. This may further obviate the need for more testing including CT imaging which exposes a subject to radiation. Furthermore, Parameter D is not influenced by age, height, and sex, obviating the need for periodic reference equations from a representative normal population.

### II. Methods

A. Data Collection

Data was collected from a group of subjects enrolled in the Genetic Epidemiology of COPD (COPDGene) study, which included current and former smokers aged 45-80 years old. Each subject was assessed for respiratory morbidity using a questionnaire and a six-minute walk test. CT images and spirometry data were additionally collected from each subject.

B. CT Metrics

Collected CT images included volumetric CT scans obtained at maximal inspiration and end-tidal expiration to indicate a total lung capacity and functional residual capacity of a subject. Emphysema and gas trapping were quantified using 3D Slicer software (Chest Imaging Platform (CIP) Boston, Mass., USA), and Apollo Software (VIDA Diagnostics, Coralville, Iowa, USA) was used to measure airway dimensions.

A classification of emphysema was determined using a measure of lung volume at total lung capacity for a subject. For example, mild emphysema was indicated by a percentage of lung volume at total lung capacity with attenuation <−910 Hounsfield Units (HU) (low attenuation area, % LAA910ᵢₙₛₚ). Severe emphysema was indicated by % LAA <−950 HU.

Gas trapping was also identified using a measure of lung volume at an end expiration for a subject. For example, a percentage of lung volume at an end expiration with attenuation less than −856 HU can be used to indicate gas trapping.

Wall area percentage of segmental airways (Wall area pct) was used to quantify airway disease. In addition, parametric response mapping was used to match inspiratory and expiratory images voxel-to-voxel. A percentage of non-emphysematous gas trapping, or functional small airways disease (PRM^{fSAD}) was calculated as a measure of small airways disease.

C. Spirometry Metrics

A reading with a highest sum of values for a forced expiratory volume within one second and a forced vital capacity was selected for analyses per the American Thoracic Society (ATS) criteria. Volume measurements were collected every 60 msec and flow measurements were collected every 30 ml in order to determine a volume-time curve and flow-volume curve for each selected reading. Individual data points in the flow-volume and volume-time curves were analyzed to quantify important transition points and contours in the expiratory curves.

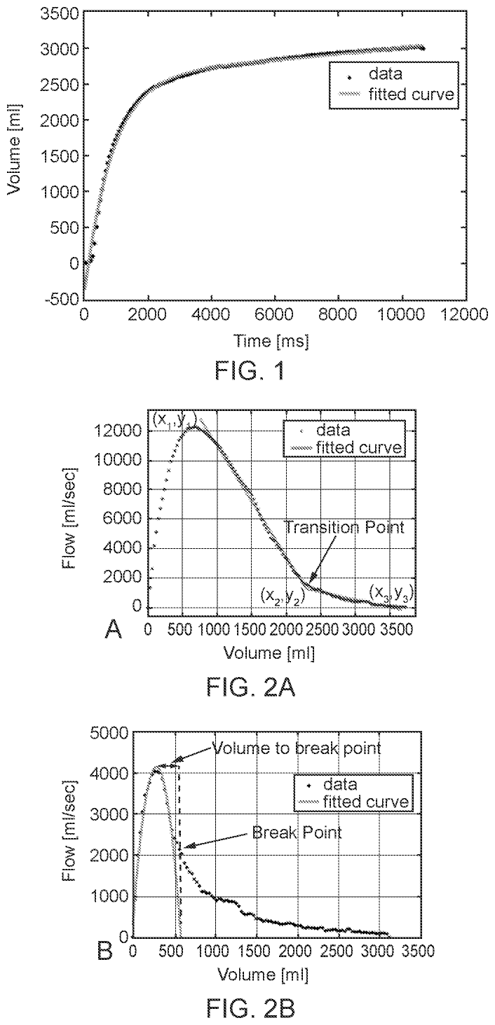

Shape of the volume-time curve. A shape of the volume-time curve was determined using a curve fitting algorithm. Initially, the curve-fitting algorithm used was Levenberg-Marquardt algorithm (LMA), which fit a model to the volume-time curve: V_{estimated}=Ae^{Bt}+Ce^{Dt}, where A, B, C, D are the parameters determined using a function fitting optimization process minimizing J=∥V_{measured}−Ae^{Bt}−Ce^{Dt}∥ cost function. To differentiate between the Ae^{Bt} and Ce^{Dt} terms, it was assumed that A>0 and C<0. The first term, Ae^{Bt} represents a rising slope of volume increase closer to the end of the exhalation, and the second term, Ce^{Dt}, describes an overall volume-time curve, where Parameter D describes a rate of volume increase. FIG. 1 shows an example of fitting a function to the volume-time curve. In embodiments, the rate of volume of increase described Parameter D may be used to identify deficiencies in subject expiration. For example, a Parameter D below some predetermined threshold value may indicate a subject with airflow obstruction.

However, LMA assumes a normal distribution of residuals (e.g., an error between an estimated point and an actual point of the volume-time curve) and does not suitably identify a curve that fits the volume-time expiratory data when a distribution of residuals is not normal. In order to address this issue, various embodiments described herein have updated Parameter D to use a least absolute residuals (LAR) algorithm for curve-fitting. Because LAR does not involve calculating a sum of squares, the algorithm does not include a same sensitivity to outliers that may be found in calculations using LMA.

Using LMA as the curve-fitting algorithm, Parameter D could only achieve curve fitting in 66% of individuals with at least 95% accuracy. However, the updated Parameter D using LAR enables curve fitting in 99.5% of individuals with 99.9% accuracy, which is a significant improvement over the previous technique for determining Parameter D.

Data was additionally adjusted in order to remove a first few data points of volume-time expiratory data to improve curve fitting. Specifically, data for a first 50 mL of expiratory data was excluded when calculating the Parameter D. Additionally, the end of the test may be modified to standardize the end point for the curve. Instead of using the last recorded point on the volume-time curve which is variable depending on when the subject was asked to quit exhaling, the highest volume recorded may be used as the end point of the curve. Alternatively, the end point of the curve may be set at a predetermined amount of time such as 6 seconds or the highest volume recorded, whichever occurs earlier.

Transition Point. A Transition Point is defined by fitting a piecewise function with two linear segments to the flow-volume curve. In particular, the piecewise function may be fit around a highest point of the flow-volume curve. In such instances, data before the peak expiratory flow may be ignored (see FIG. 2A). A nonlinear least-squares algorithm was used to find the optimal fit parameters of the curve (x₁, y₁), (x₂, y₂), (x₃, y₃). The Transition Point is defined as x₂, which is an intersection point for the two linear segments. The Transition Point may not be easily identified in every instance (e.g., even with the aid of computational tools) because a plurality of slopes corresponding to the flow-volume curve may not fit on linear regression lines.

Transition Distance. Transition distance may be another metric for calculating how quickly a plurality of slopes may change. In order to calculate the Transition Distance, an inverted parabola may be fitted around a peak point of the flow-volume curve using a least squares minimization algorithm as shown in FIG. 2B. A breaking point between the parabola and the remainder of the flow-volume curve is defined as a latest sample that still provides a goodness of fit of at least R²>0.96. The Transition Distance is defined as the distance on the X-axis (in ml) from the peak of the fitted parabola to the breaking point (see FIG. 2B).

D. Analysis

A presence of COPD was defined by a FEV₁/FVC ratio being below a first threshold value of 0.70. Subjects were excluded with Preserved Ratio Impaired SpiroMetry (PRISm, FEV₁/FVC>0.70 but FEV₁<80% predicted) to avoid confounding by restrictive processes. Data was further collected from a population of non-smoker subjects and used to calculate a second threshold value (e.g., a 90th or 75th percentile of normal) for Parameter D. The second threshold value was found to be −0.104 using LMA. Subjects with a Parameter D less than this second threshold value were deemed to have an abnormality in Parameter D (e.g., the rate of volume increase).

Abnormalities within a combination of a FEV₁/FVC ratio and Parameter D were then used to determine diagnoses for subjects. For example, a subject with both a positive (e.g., below a first threshold value) FEV₁/FVC ratio and a positive (e.g., below a second threshold value) Parameter D is defined as having COPD. A subject with a negative (e.g., equal to or above the first threshold value) FEV₁/FVC ratio and a negative (e.g., equal to or above the second threshold value) Parameter D is deemed to have no airflow obstruction. A subject with a positive Parameter D but negative FEV₁/FVC ratio may be categorized as having Discordant COPD, which represents additional subjects with airflow obstruction detected using Parameter D. Comparisons were repeated with COPD defined by FEV₁/FVC <5th percentile of predicted value for age, sex, race and height (lower limit of normal, LLN) as having COPD-LLN. In further instances, a subject below a 10th percentile of a third threshold value for Transition Point (17.0) and a fourth threshold value for Transition Distance (30.0) can also be identified as having airflow obstruction.

Receiver operating characteristic (ROC) analyses measured the accuracy of the novel spirometry metrics in comparison with FEV₁/FVC for identifying thresholds of structural lung disease on CT (5% severe emphysema and 5% functional small airway disease or fSAD). Generalized linear regression models were used to test associations between the novel spirometry metrics and structural lung disease as well as respiratory morbidity indices. To assess performance of the novel metrics in those with mild disease, characteristics were compared for those with GOLD stage 0 and 1 only, and tested concordance for diagnosis using FEV₁/FVC <0.70 (or <LLN) versus abnormal spirometry by novel indices. Comparisons were made between subjects categorized as concordant and discordant for airflow obstruction by traditional and novel spirometry metrics with subjects belonging to a “smoker” category identified as concordant for not having airflow obstruction, using Analysis of Variance (ANOVA). Because smokers identified as concordant were used as a reference group, adjusted odds ratios for CT measures of structural lung disease were estimated in each group. Cox proportional hazards were calculated for mortality for each higher quartile of Parameter D with a lowest quartile (Q1) as the reference representing subjects classified as concordant. Statistical significance was set at a two-sided alpha of 0.05. All analyses were performed using Statistical Package for the Social Sciences (SPSS 24.0, SPSS Inc., Chicago, Ill., USA).

### III. Results

Performance of metrics was examined in a population of 8307 subjects with a full set of spirometry and CT imaging data. Mean age of subjects was 60.0 (with a standard deviation of 9.1) years. The population of subjects comprised of 45.5% females and 31.1% African Americans. Parameter D, Transition point and Transition Distance could be calculated in 5532 (66.6%), 7960 (95.8%), and 7960 (95.8%) of expiratory curves respectively generated for each subject. Parameter D ranged from −0.41 to 0.02, with more positive values indicating greater disease; Transition point ranged from 4.0 to 133.0 and Transition Distance ranged from 30.0 to 2220.0, where lower values of the Transition Point and/or Transition Distance indicated greater disease. Subjects encompassed a range of severity of airflow obstruction with 49.5%, 9.1%, 21.9%, 13.0%, and 6.5% with Global Initiative for Chronic Obstructive Lung Disease (GOLD) stages 0 through 4, respectively. Calculating Parameter D became progressively more difficult as a rate of disease worsened and could be calculated in 82%, 75%, 58%, 37% and 28%, respectively of subjects with GOLD stages 0 through 4. As more severe disease is easily detected using traditional spirometry criteria, a second stage of analysis was performed to detect mild airflow obstruction (See section III.B). Overall, analysis of the population of subjects indicated significant correlations between FEV1/FVC and FEV1% predicted with Parameter D (r=−0.83; p<0.001 and −0.66; p<0.001, respectively), Transition Point (r=0.69; p<0.001 and 0.71; p<0.001, respectively) and Transition Distance (r=0.50; p<0.001 and 0.52; p<0.001, respectively).

A. Association of Novel Metrics with Structural Lung Disease

Parameter D and FEV1/FVC were similar in accuracy for identifying >5% severe emphysema (% LAA<−950 HU) (c-statistic 0.83, 95% CI 0.82-0.84; p<0.001, and 0.83, 95% CI 0.81-0.84; p<0.001, respectively), whereas the c-statistic for Transition Point, Transition Distance, and FEV1% predicted were 0.71 (95% CI 0.70-0.73; p<0.001), 0.68 (95% CI 0.66-0.69; p<0.001), and 0.73 (95% CI 0.71-0.75; p<0.001), respectively. Parameter D and FEV1/FVC also had a comparable accuracy for identifying 10% severe emphysema (c-statistic 0.91, 95% CI 0.89-0.92; p<0.001, and 0.91, 95% CI 0.90-0.93; p<0.001, respectively). For 10% emphysema, Transition Point, Transition Distance and FEV1% predicted had improved accuracies with a c-statistic of 0.81 (95% CI 0.79-0.83; p<0.001), 0.77 (95% CI 0.75-0.79; p<0.001), and 0.84 (95% CI 0.82-0.86; p<0.001), respectively.

Parameter D and FEV1/FVC were similar in accuracy for identifying >5% fSAD (e.g., small airway disease) with a c-statistic of 0.76, 95% CI 0.74-0.78; p<0.001, and 0.78, 95% CI 0.77-0.80; p<0.001, respectively, whereas the c-statistic for Transition Point, Transition Distance, and FEV1% predicted were 0.63 (95% CI 0.61-0.64; p<0.001), 0.59 (95% CI 0.57-0.61; p<0.001), and 0.66 (95% CI 0.65-0.68; p<0.001), respectively.

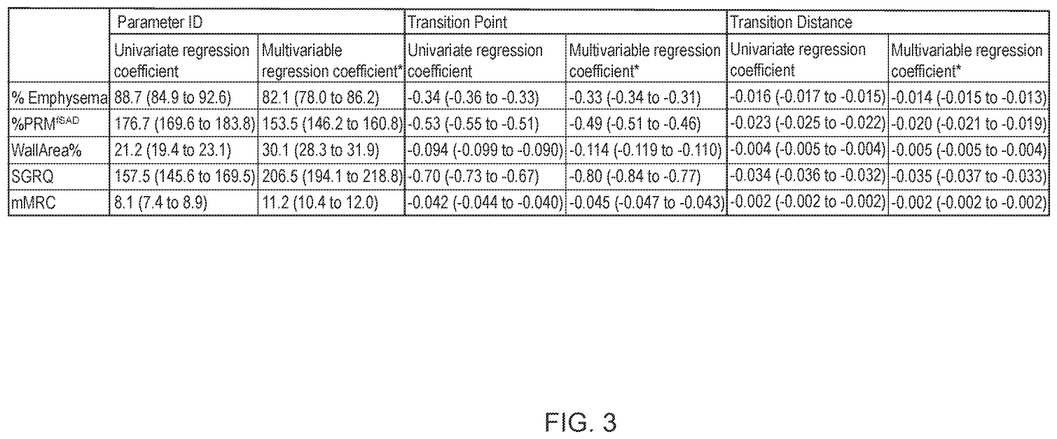

All novel metrics had significant associations with emphysema, small airway disease, medium size airway disease, as well as respiratory morbidity, after adjustment for age, sex, race, BMI, and scanner type for CT parameters (FIG. 3).

B. Association with Mortality

B.1. Parameter D Using LMA

Follow-up data was collected on 7294 subjects for a median (interquartile range, IQR) of 6.6 (5.8 to 7.3) years. 993 (12.0%) subjects died on follow-up. The follow-up data was available in 4843 (88%) of subjects and a Parameter D value was initially calculated using LMA. Subjects were categorized into quartiles of Parameter D, the higher two quartiles (≥−0.082 and −0.113 to −0.082) were associated with greater mortality compared with the lowest quartile (≤−0.142), unadjusted hazards ratio, HR 4.47, 95% CI 3.42-5.85; p<0.001 and 1.41, 95% CI 1.03-1.93; p=0.031, respectively. After adjustment for age, sex, race and body-mass-index (BMI), only the highest quartile was significantly associated with mortality compared to the lowest quartile (adjusted HR 3.22, 95% CI 2.42-4.27; p<0.001). There were a substantial number of subjects in the highest quartile of Parameter D who were categorized as GOLD 0 and 1 by traditional criteria (1.8% and 33.7%, respectively). On the other hand, 90.3% and 99.3% of GOLD 3 and 4 in whom Parameter D could be measured were comprised of subjects in the highest quartile of Parameter D.

B.2. Updated Parameter D using LAR

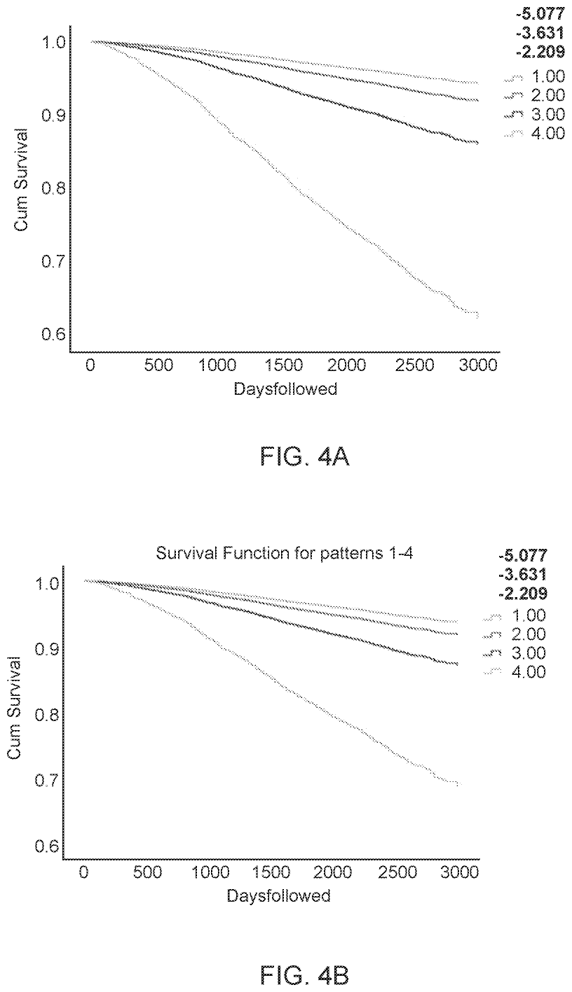

Updated Parameter D values were later calculated for subjects in order to identify a mortality rate of each subject based on the updated Parameter D value associated with the subject. Subjects were stratified by quartiles of the updated Parameter D, where each quartile indicated progressive greater hazards of mortality based on a higher Parameter D value. FIG. 4A shows a plot of an unadjusted analysis with a Y-axis indicating a cumulative rate of survival and a X-axis indicating a time period. The plot further illustrates quartiles of Parameter D and the cumulative rate of survival for subjects within each quartile (all p<0.001). Quartiles were defined for the New parameter D as Parameter D value of a normal subject that is less than −5.077; (ii) a second quartile that corresponds to an average Parameter D value between −5.076 and −3.631; (iii) a third quartile corresponds to an average Parameter D value between −3.630 to −2.209; and a fourth quartile corresponds to an average Parameter D value equal to or greater than −2.209. Unadjusted survival analyses showed progressively higher mortality with higher quartiles, hazards ratio (HR) for Q2-Q4: Q2: 1.44, 95% CI 1.08-1.92; HR 2.56, 95% CI 1.97-3.32; and HR 8.02, 95% CI 6.32-10.16, respectively (FIG. 4A).

After adjustment for age, sex, race, and BMI, the quartiles remained associated with higher mortality with worsening quartile; adjusted HR for Q2-4 were 1.32, 95% CI 0.99-1.76, 2.10, 95% CI 1.61-2.74, and 5.73, 95% CI 4.47-7.34, respectively (FIG. 4B).

C. Mild Disease

A second stage of analysis focused on 4870 subjects with GOLD stage 0 and 1. Mean age was 57.5 (SD 8.6) years, and the subset was comprised of 46.4% females, and 37.7% African Americans. Both the Transition Point and Transition Distance could be calculated in 4686 (96.2%) of expiratory curves whereas Parameter D could be calculated in 3930 (80.7%). 760 out of 4870 (15.6%) subjects had airflow obstruction by traditional GOLD criteria and 445 out of 4870 (9.1%) subjects had airflow obstruction using the LLN criteria for FEV1/FVC. 873 out of 3930 (17.9%) subjects had airflow obstruction per Parameter D. 721 out of 4686 (14.8%) subjects had airflow obstruction per Transition Point. 788 out of 4612 (16.2%) subjects had airflow obstruction per Transition Distance.

C.1. Parameter D Using LMA

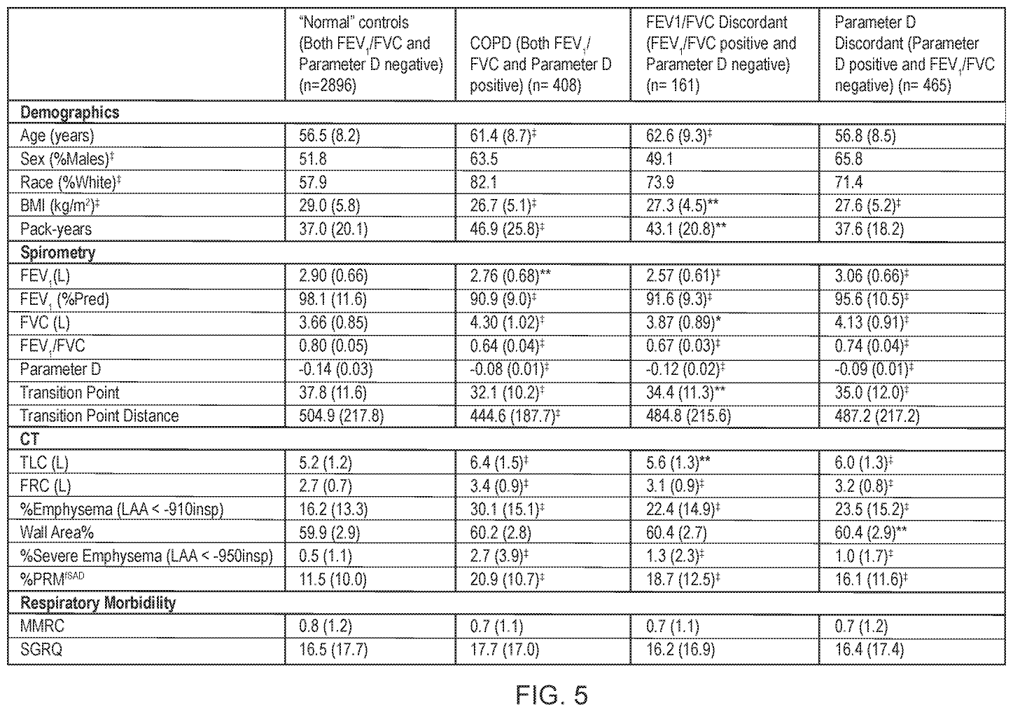

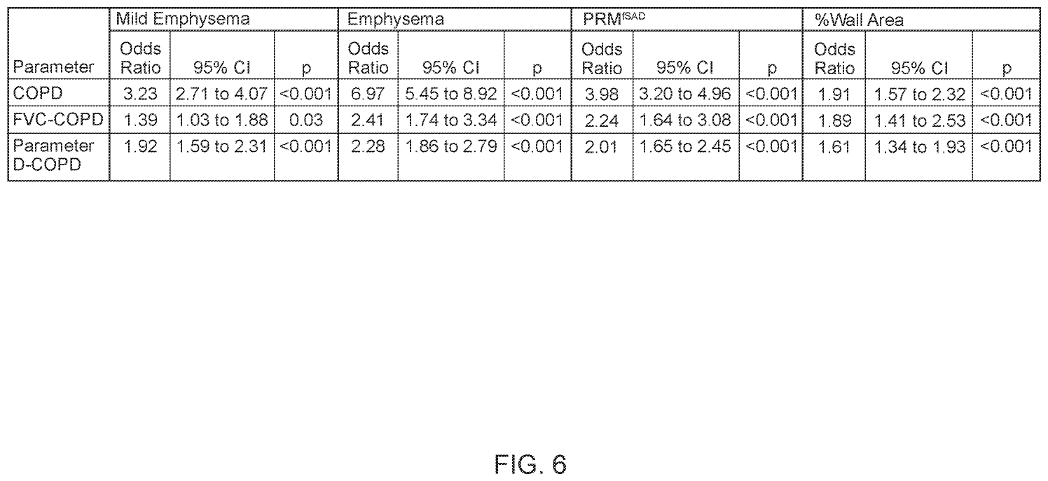

FIG. 5 shows a comparison of subjects classified as concordant and discordant for abnormality by both FEV₁/FVC <0.70 and a Parameter D value, wherein the Parameter D value was calculated using LMA. In comparison to traditional spirometry criteria, Parameter D (using LMA) identified an additional 9.5% of subjects with mild or non-recognized disease as abnormal, and this proportion was 11.8% where Parameter D was calculable. Compared with subjects who were classified as concordant normal, the subjects classified as discordant based on only a Parameter D were similar in age but with higher FEV1 and FVC as well as CT total lung capacity and FRC, but had higher CT measures of emphysema, functional small airway disease as well as segmental bronchial wall thickness. These relationships held true after adjusting for age, sex, race, BMI, and CT scanner type (see FIG. 6). Of the 465 discordant cases positive by Parameter D alone, more subjects had emphysema >5% (20.0% vs. 8.9%; p<0.001) and PRMfSAD>15% (43.4% vs. 26.5%; p<0.001), compared with concordant normals. Of those positive by Parameter D alone, 115 (24.7%) and 91 (19.6%) had substantial symptoms as evidenced by St. George's Respiratory Questionnaire (SGRQ) score >25 and modified Medical Research Council (mMRC) dyspnea score>2, respectively.

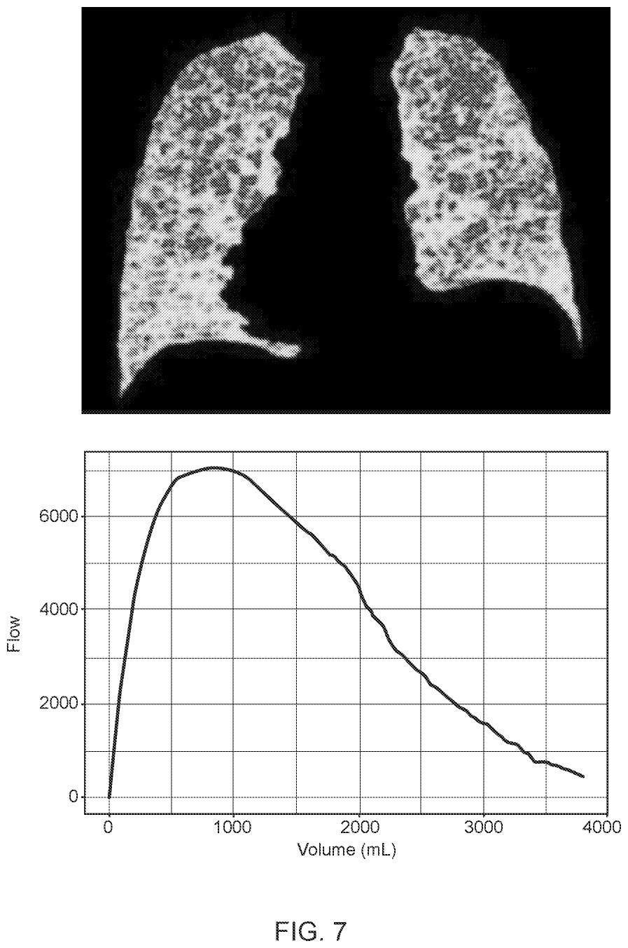

FIG. 7 shows a representative subject not detected by traditional criteria but displaying an abnormal Parameter D. Subject indicated significant symptom burden, with mMRC score of 3, and SGRQ score of 48. However, lung function for the subject by traditional criteria was normal with FEV1/FVC of 0.72, and FEV1% predicted of 100.1%. A flow-volume curve determined for the subject appears normal, however Parameter D was −0.08, which is outside of a normal threshold. A CT image of the subject's lungs revealed 0.5% emphysema and 25% fSAD.

C.2. Updated Parameter D Using LAR

An updated Parameter D value was additionally calculated using post bronchodilator curves and LAR instead of LMA as described herein. Responsive to using the updated Parameter D for detection of mild disease, a new 90^{th} percentile (−4.083) was defined as a new threshold. Updated Parameter D values were then able to detect an additional 17% individuals who were initially classified as normal by traditional criteria (e.g., a FEV₁/FVC ratio less than 0.70) using this new threshold.

Subsequently, data was collected from a group of subjects enrolled in the National Health and Nutrition Examination Survey (NHANES) study. An updated Parameter D value was calculated using pre bronchodilator curves (post not available here) and LAR. Responsive to using the updated Parameter D for detection of mild disease, a new 75^{th} percentile (−3.261) was defined as a new threshold. Table 1 shows 75^{th} percentile threshold derived from NHANES (−3.261) applied to data from the COPDGene study to compare agreement of the updated Parameter D with traditional FEV1/FVC based on LLN.

TABLE 1

| | | | Lower limit of | |
|---|---|---|---|---|
| | Both | Parameter D | normal alone | Both |
| Classification | Negative | only positive | positive | positive |
| n | 4075 | 1494 | 234 | 3882 |
| % | 42.1 | 15.4 | 2.4 | 37.1 |

Thus, the updated Parameter D identifies an additional 15.4% subjects with airflow obstruction while missing only 2.4% detected by traditional metrics.

Table 2 shows a comparison of structural disease on CT using updated Parameter D versus traditional LLN classification.

TABLE 2

| | Normal | D + COPD | D − COPD | COPD |
|---|---|---|---|---|
| | n = 4075 | n = 1494 | n = 234 | n = 4075 |
| | (42.1%) | (15.4%) | (2.4%) | (42.1%) |
| % Emphy- | 369 (10%) | 229 (17%) | 32 (15%) | 2182 (60%) |
| sema ≥5 | | | | |
| % fSAD ≥15 | 401 (12%) | 294 (25%) | 42 (23%) | 2363 (74%) |

D. Additional Analyses

It will further be appreciated that the updated Parameter D calculated using LAR, rather than LMA is not affected by an age of a subject and/or a height of a subject, and/or a sex of a subject.

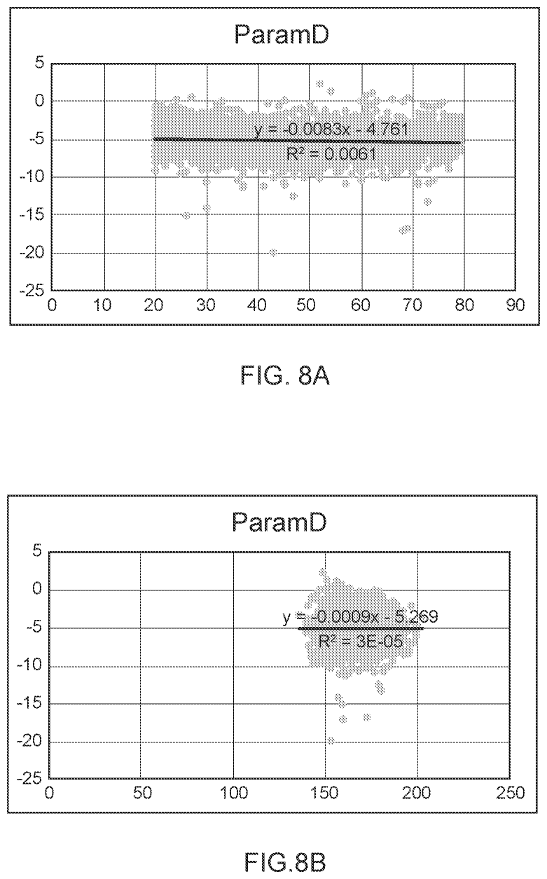

FIGS. 8A-8B show plots associating Parameter D with age and height data for a population of asymptomatic 5,030 subjects. Subjects ranged from ages of 20-79 years old. FIG. 8A shows a plot correlating a Parameter D for each subject with an age (e.g., in years) of each subject. The data can be represented by y=−0.0083x−4.761 with R²=0.0061.

FIG. 8B shows another plot correlating the Parameter D for each subject with a height of each subject. Height was calculated in cm. Plot data indicated a line y=0.0009x−5.269 with R²=3E−5.

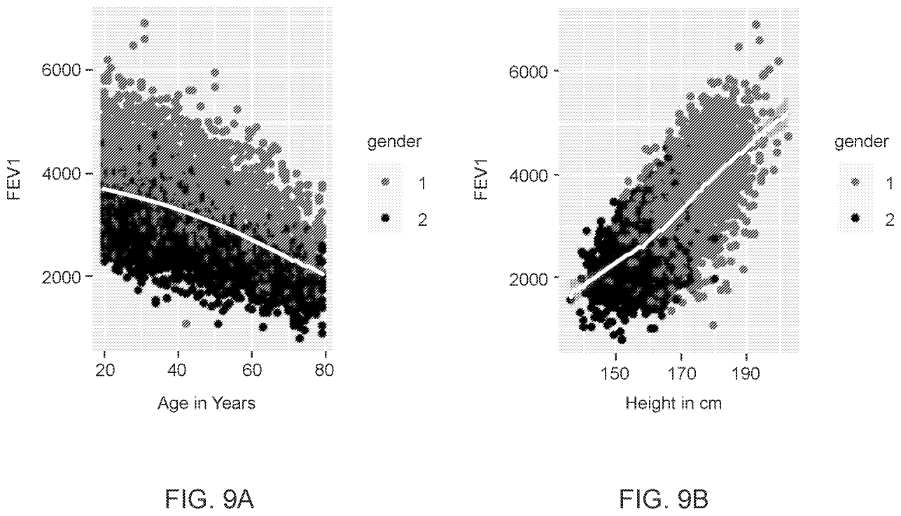

As a comparison, FIGS. 9A-9B show plots associating a FEV1 value with age, height, and sex data for a population of subjects from the NHANES study. FIG. 9A shows a plot correlating a FEV1 value associated with each subject to an age of each respective subject. FIG. 9B shows another plot correlating a FEV1 value associated with each subject to a height of each respective subject. Genders of subject were also differentiated for each plot (Gender 1=male; and Gender 2=female). Both plots indicate significant correlations between subject age, height, and sex and the calculated FEV1 values. For example, older subjects appear to be associated with lower FEV1 values.

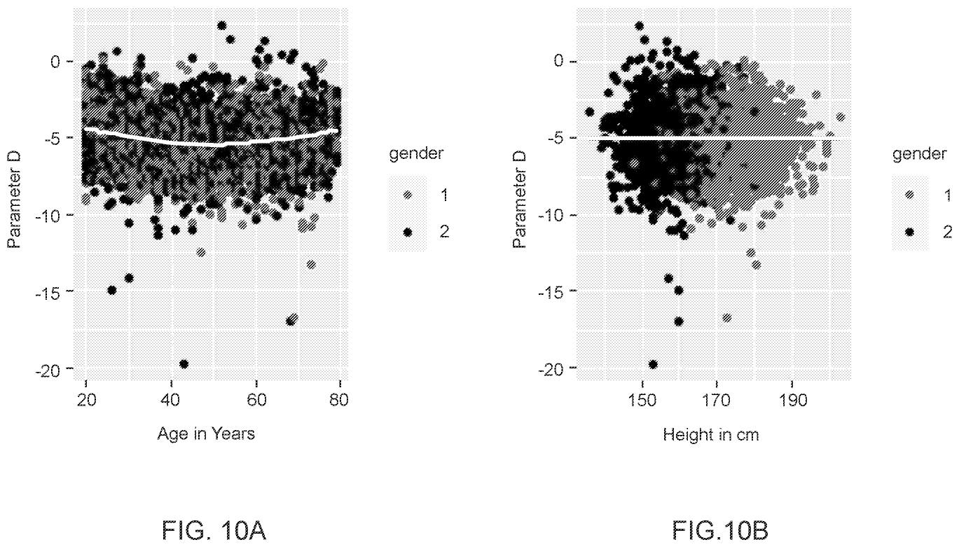

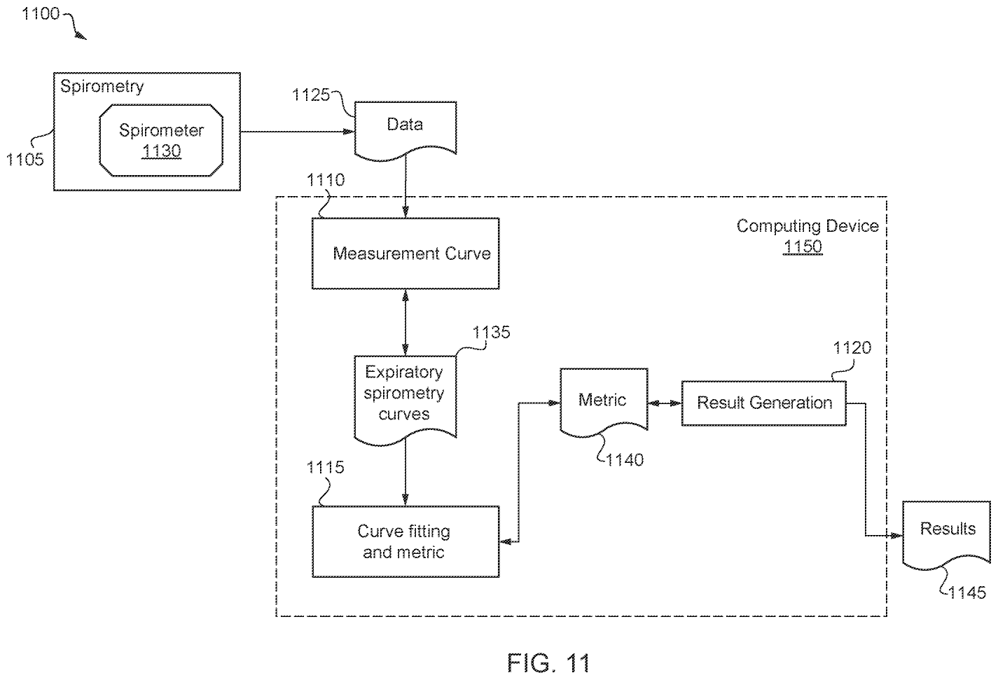

FIGS. 10A-10B additionally show plots associating Parameter D with age, height, and sex data for the same population of subjects as FIGS. 9A-9B. FIG. 10A shows a plot correlating a Parameter D associated with each subject to an age of each respective subject. FIG. 10B shows another plot correlating a Parameter D associated with each subject to a height of each respective subject. Genders of subject were again differentiated for each respective plot. Unlike the FEV1 values for each subject, the plots show there is no significant correlation between an age, height, and/or sex of a subject and a Parameter D calculated for the subject.

This is particularly advantageous because traditional metrics of measuring lung function, such as calculating a FEV1, a FVC, and/or a FEV1/FVC ratio, are often influenced by characteristics of a subject like age, sex, and/or height.

### IV. Discussion

Traditional spirometry criteria are simple to use and perform well in detecting more apparent disease, but fail to diagnose a number of mild cases. In particular, the qualitative assessment of expiratory curves focuses on analyzing differences that may not be readily apparent until the disease is far advanced. Failure to diagnose mild cases at an earlier stage of the disease often hinders a longevity and quality of life of subjects.

In order to address this issue, novel spirometry metrics were derived for each subject from a population of current and former smokers. Rather than analyzing only fixed portions of the expiratory flow-volume curves, individual data points are analyzed to determine one or more of these novel metrics. These novel metrics may then be used to identify additional subjects with structural and clinical lung disease that had initially been diagnosed as normal and/or concordant (e.g., not having a disease) by one or more traditional criteria. CT imaging was used on subjects in order to identify a presence or absence of at least structural lung disease. Results of CT images were then used to verify an accuracy of predictions made by the novel metrics. It was found that the novel metrics performed with a higher sensitivity in detecting airflow obstructions, particularly mild airflow obstructions, in comparison to traditional spirometry criteria. The novel metrics can additionally be used to identify subjects with a high risk of mortality.

These novel metrics can be easily adapted into commercially available spirometry software without any change in testing procedures to provide additional outputs that can help inform the likelihood of airflow obstruction in borderline cases. In cases where volume-time curves or flow-volume curves are sampled at different frequencies, the curves can be resampled at the same rate as described herein.

Embodiments of the present disclosure further include several advantages with its use of Parameter D. Since Parameter D can represent a slow exponential decay in volume over a later part of the volume-time curve, the metric is likely a reflection of small airway involvement and changes in elastic recoil of the lung. The metric is further shown (e.g., via image matching) as strongly associated with PRM^{fSAD}, a measure of non-emphysematous gas trapping. For at least these reasons, Parameter D was used to identify a substantial number of additional (e.g., asymptomatic and symptomatic) subjects that may otherwise have been excluded and/or classified as concordant based on traditional spirometry criteria.

Using LAR over LMA as the mathematical function for curve fitting used to calculate Parameter D further increased a rate of curve fitting for subjects and was used to determine a new threshold value for Parameter D. The new threshold value was used to identify an additional percentage of subjects that had been previously undiagnosed, which further increased a sensitivity of airflow obstruction detection. Prior methods using LMA additionally could not be assessed in those with very severe cases of disease, but the use of LAR can be used to identify both severe and mild cases.

### V. Systems and Methods for Determining a Presence or Absence of Airflow Obstruction

FIG. 11 shows a block diagram that illustrates a computing environment 1100 for obtaining and processing spirometry data. As further described herein, obtaining and processing spirometry data can include generating measurement curves, performing curve fitting on the curves using functions or algorithms to determine various metrics, and use those various metrics to determine a presence or absence of airflow obstruction for the subject.

As shown in FIG. 11, computing environment 1100 includes several stages: a spirometry stage 1105, a measurement curve stage 1110, a curve fitting and metric stage 1115, and a result generation stage 1120. The spirometry stage 1105 includes obtaining data 1125 corresponding to one or more expiratory air measurements for a subject. The one or more expiratory air measurements are obtained using a spirometer 1130. A spirometer is a device for measuring the volume and flow of air inspired and expired by the lungs.

The measurement curve stage 1110 includes generating expiratory spirometry curves 1135 based on the obtained data 1125 for the subject. Expiratory spirometry curves may include at least a volume-time curve illustrating a volume of air exhaled over a time period and a flow-volume curve illustrating a rate of air flow over a volume of air exhaled. For example, the spirometer 1130 may be used to collect volume measurements every 60 msec and flow measurements every 30 ml in order to generate a volume-time curve and flow-volume curve for each selected reading.

The curve fitting and metric stage 1115 deriving spirometry metrics 1140 from the Expiratory spirometry curves 1135. The spirometry metrics 1140 are generated by modelling expiratory spirometry curves 1135 using a mathematical function using a function-fitting process. For example, a Least Absolute Residuals function may be applied to the volume-time curve to estimate a function which closely approximates the obtained data 1125 for the subject by minimizing a sum of absolute deviation; and a metric (i.e., shape of the volume-time curve) that describes a rate of volume increase may be determined based on the estimated function. Further, a piece-wise function with at least two or more linear segments may be fit around a highest point of the flow-volume curve (a nonlinear least-squares algorithm may be used to find the optimal fit parameters of the curve); and a metric (i.e., transition point) may be determined based on an intersection point for the at least two or more linear segments of the piece-wise function. Further, an inverted parabola may be fitted around a peak point of the flow-volume curve using a least squares minimization algorithm; and a metric (i.e., transition distance) may be determined from the highest point of the flow-volume curve and a point where the applied function deviates from the flow-volume curve. As used herein, when an action is “based on” something, this means the action is based at least in part on at least a part of the something.

The result generation stage 1120 includes comparing the metrics 1140 to threshold values and determining a result 1145 such as the presence or absence of airflow obstruction for the subject based on the comparison. In some instances, the result 1145 may further pertain to a determination that the presence of the airflow obstruction is associated with COPD based on the comparison. In some instances, the result may further pertain to a determination of a cumulative rate of survival for the subject based on the comparison.

The measurement curve stage 1110, the curve fitting and metric stage 1115, and the result generation stage 1120 may be implemented using software (e.g., code, instructions, program) executed by one or more processing units (e.g., processors, cores) of respective systems, hardware, or combinations thereof. The respective systems, hardware, or combinations may include the spirometer 1135 and/or computing device 1150. For example, the software executed by one or more processing units may reside in the spirometer 1135, the computing device 1150, or a combination thereof.

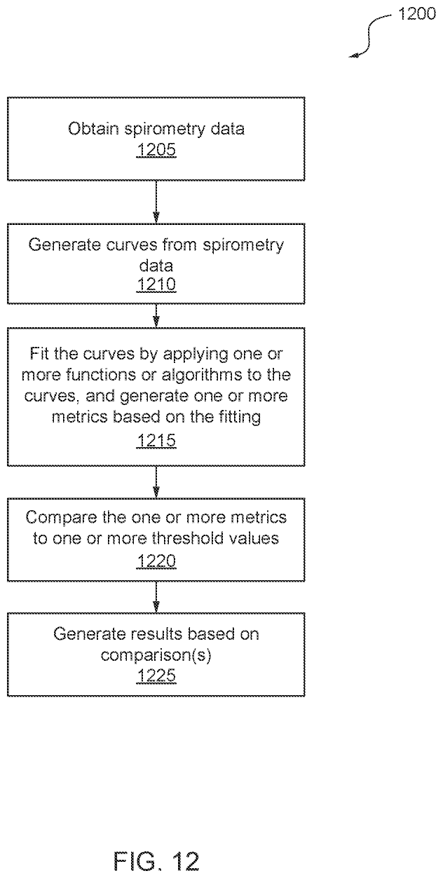

FIG. 12 shows a flowchart illustrating a process 1200 for obtaining and processing spirometry data to determine a presence or absence of airflow obstruction for a subject in accordance with various embodiments. The process 1200 depicted in FIG. 12 may be implemented in software (e.g., code, instructions, program) executed by one or more processing units (e.g., processors, cores) of the respective systems, hardware, or combinations thereof. The software may be stored on a non-transitory storage medium (e.g., on a memory device). The process 1200 presented in FIG. 12 and described below is intended to be illustrative and non-limiting. Although FIG. 12 depicts the various processing steps occurring in a particular sequence or order, this is not intended to be limiting. In certain alternative embodiments, the steps may be performed in some different order or some steps may also be performed in parallel. In certain embodiments, such as in the embodiments depicted in FIG. 11, the processing depicted in FIG. 12 may be performed by a computing device or system (e.g., spirometer 1135, the computing device 1150, or a combination thereof) to determine a presence or absence of airflow obstruction for a subject.

Process 1200 starts at block 1205, at which data corresponding to one or more expiratory air measurements for a subject are obtained using a spirometer.

At block 1210, a first measurement curve and a second measurement curve are generated based on the obtained data for the subject. In some instances, the first measurement curve indicates a volume of air exhaled over a time period (i.e., volume-time curve) and the second measurement curve indicates a rate of flow over a volume of air exhaled (i.e., flow-volume curve).

At block 1215, at least a first curve-fitting is performed on the first measurement curve. The first curve-fitting on the first measurement curve may be performed by: (i) applying a first function (includes Least Absolute Residuals) to the first measurement curve to estimate a function which closely approximates the obtained data for the subject by minimizing a sum of absolute deviation, and (ii) determining a first metric based on the estimated function, where the first metric describes a rate of volume increase. Optionally, at least a second curve-fitting is performed on the second measurement curve. The second curve-fitting on the second measurement curve may be performed by: (i) applying a second function with at least two or more linear segments to a portion of the second measurement curve, and (ii) determining a second metric based on an intersection point for the at least two or more linear segments of the piece-wise function, where the second metric describes a transition point. Optionally, at least a third curve-fitting is performed on the second measurement curve. The second curve-fitting on the second measurement curve may be performed by: (i) applying a third function around a highest point of the second measurement curve using a least squares minimization, and (ii) determining a third metric from the highest point of the second measurement curve and a point where the applied function deviates from the second measurement curve, where the third metric describes a transition distance. As used herein, the terms “substantially,” “approximately,” “around,” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

At block 1220, the first metric is compared to one or more threshold values. Optionally, the first metric, the second metric, and the third metric are compared to a plurality of threshold values. In some instances, the first metric is compared to: (i) a first quartile that corresponds to a reference Parameter D value of a normal subject that is less than −5.077; (ii) a second quartile that corresponds to an average Parameter D value between −5.076 and −3.631; (iii) a third quartile that corresponds to an average Parameter D value between −3.630 and −2.209; and a fourth quartile that corresponds to an average Parameter D value equal to or greater than −2.209.

At block 1225, a result is generated based on the comparison of the first metric to the one or more threshold values. In some instances, a presence or absence of airflow obstruction for the subject is determined based on the comparison of the first metric to the one or more threshold values. In other instances, a presence or absence of airflow obstruction for the subject is determined based on the comparison of the first metric, the second metric, and the third metric to the plurality of threshold values. In some instances, a cumulative rate of survival is determined for the subject based on the comparison, where subjects within the fourth quartile have a lower cumulative rate of survival as compared to subjects in the first-third quartile. In some instances, the presence of the airflow obstruction is determined to be associated with COPD; and a treatment plan is generated and implemented for COPD for the subject based on the determined presence of the airflow obstruction and/or the determined cumulative rate of survival for the subject.

### VI. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

1. A method comprising:
obtaining, using a spirometer, data corresponding to one or more expiratory air measurements for a subject;
generating a first measurement curve and a second measurement curve based on the obtained data for the subject;
performing at least a first curve-fitting on the first measurement curve, wherein the performing the first curve-fitting on the first measurement curve comprises:
applying a first function to the first measurement curve to estimate a function which closely approximates the obtained data for the subject by minimizing a sum of absolute deviation, wherein the first function includes Least Absolute Residuals; and
determining a first metric based on the estimated function, wherein the first metric describes a rate of volume increase;
comparing the first metric to one or more threshold values; and
determining a presence or absence of airflow obstruction for the subject based on the comparison of the first metric to the one or more threshold values.

2. The method of claim 1, wherein the comparing the first metric comprises:
comparing the first metric to: (i) a first quartile that corresponds to a reference Parameter D value of a normal subject that is less than −5.077; (ii) a second quartile that corresponds to an average Parameter D value between −5.076 and −3.631; (iii) a third quartile that corresponds to an average Parameter D value between −3.630 and −2.209; and a fourth quartile that corresponds to an average Parameter D value equal to or greater than −2.209; and
determining a cumulative rate of survival for the subject based on the comparison, wherein subjects within the fourth quartile have a lower cumulative rate of survival as compared to subjects in the first-third quartile.

3. The method of claim 2, wherein the first measurement curve indicates a volume of air exhaled over a time period and the second measurement curve indicates a rate of flow over a volume of air exhaled.

4. The method of claim 1, further comprising determining the presence of the airflow obstruction is associated with chronic obstructive pulmonary disease (COPD); and generating and implementing a treatment plan for COPD for the subject based on the determined presence of the airflow obstruction and/or the determined cumulative rate of survival for the subject.

5. The method of claim 1, further comprising:
performing at least a second curve-fitting on the second measurement curve, wherein the performing the second curve-fitting on the second measurement curve comprises:
applying a second function with at least two or more linear segments to a portion of the second measurement curve; and
determining a second metric based on an intersection point for the at least two or more linear segments of the piece-wise function;
performing at least a third curve-fitting on the second measurement curve, wherein the performing the third curve-fitting on the second measurement curve further comprises:
applying a third function around a highest point of the second measurement curve using a least squares minimization; and
determining a third metric from the highest point of the second measurement curve and a point where the applied function deviates from the second measurement curve;
comparing the first metric, the second metric, and the third metric to a plurality of threshold values; and
determining the presence or absence of airflow obstruction for the subject based on the comparison of the first metric, the second metric, and the third metric to the plurality of threshold values.

6. The method of claim 7, wherein the second function is a piecewise function, and the third function is an inverted parabola.

7. The method of claim 1, wherein the one or more thresholds comprise a 90^{th} percentile threshold of −4.083 or a 75^{th} percentile threshold of −4.083, and the presence of airflow obstruction for the subject is determined when the first metric is less than the 90^{th} percentile threshold of −4.083 or the 75^{th} percentile threshold of −3.261.

8. A system comprising:
one or more data processors; and
a non-transitory computer readable storage medium containing instructions which, when executed on the one or more data processors, cause the one or more data processors to perform actions including:
obtaining, using a spirometer, data corresponding to one or more expiratory air measurements for a subject;
generating a first measurement curve and a second measurement curve based on the obtained data for the subject;
performing at least a first curve-fitting on the first measurement curve, wherein the performing the first curve-fitting on the first measurement curve comprises:
applying a first function to the first measurement curve to estimate a function which closely approximates the obtained data for the subject by minimizing a sum of absolute deviation, wherein the first function includes Least Absolute Residuals; and
determining a first metric based on the estimated function, wherein the first metric describes a rate of volume increase;
comparing the first metric to one or more threshold values; and
determining a presence or absence of airflow obstruction for the subject based on the comparison of the first metric to the one or more threshold values.

9. The system of claim 8, wherein the comparing the first metric comprises:
comparing the first metric to: (i) a first quartile that corresponds to a reference Parameter D value of a normal subject that is less than −5.077; (ii) a second quartile that corresponds to an average Parameter D value between −5.076 and −3.631; (iii) a third quartile that corresponds to an average Parameter D value between −3.630 and −2.209; and a fourth quartile that corresponds to an average Parameter D value equal to or greater than −2.209; and
determining a cumulative rate of survival for the subject based on the comparison, wherein subjects within the fourth quartile have a lower cumulative rate of survival as compared to subjects in the first-third quartile.

10. The system of claim 9, wherein the first measurement curve indicates a volume of air exhaled over a time period and the second measurement curve indicates a rate of flow over a volume of air exhaled.

11. The system of claim 8, wherein the actions further include determining the presence of the airflow obstruction is associated with chronic obstructive pulmonary disease (COPD); and generating and implementing a treatment plan for COPD for the subject based on the determined presence of the airflow obstruction and/or the determined cumulative rate of survival for the subject.

12. The system of claim 8, wherein the actions further include:
performing at least a second curve-fitting on the second measurement curve, wherein the performing the second curve-fitting on the second measurement curve comprises:
applying a second function with at least two or more linear segments to a portion of the second measurement curve; and
determining a second metric based on an intersection point for the at least two or more linear segments of the piece-wise function;
performing at least a third curve-fitting on the second measurement curve, wherein the performing the third curve-fitting on the second measurement curve further comprises:
applying a third function around a highest point of the second measurement curve using a least squares minimization; and
determining a third metric from the highest point of the second measurement curve and a point where the applied function deviates from the second measurement curve;
comparing the first metric, the second metric, and the third metric to a plurality of threshold values; and
determining the presence or absence of airflow obstruction for the subject based on the comparison of the first metric, the second metric, and the third metric to the plurality of threshold values.

13. The system of claim 12, wherein the second function is a piecewise function, and the third function is an inverted parabola.

14. The system of claim 8, wherein the one or more thresholds comprise a 90^{th} percentile threshold of −4.083 or a 75^{th} percentile threshold of −4.083, and the presence of airflow obstruction for the subject is determined when the first metric is less than the 90^{th} percentile threshold of −4.083 or the 75^{th} percentile threshold of −3.261.

15. A computer-program product tangibly embodied in a non-transitory machine-readable storage medium, including instructions configured to cause one or more data processors to perform actions including:
obtaining, using a spirometer, data corresponding to one or more expiratory air measurements for a subject;
generating a first measurement curve and a second measurement curve based on the obtained data for the subject;
performing at least a first curve-fitting on the first measurement curve, wherein the performing the first curve-fitting on the first measurement curve comprises:
applying a first function to the first measurement curve to estimate a function which closely approximates the obtained data for the subject by minimizing a sum of absolute deviation, wherein the first function includes Least Absolute Residuals; and
determining a first metric based on the estimated function, wherein the first metric describes a rate of volume increase;
comparing the first metric to one or more threshold values; and
determining a presence or absence of airflow obstruction for the subject based on the comparison of the first metric to the one or more threshold values.

16. The computer-program product of claim 15, wherein the comparing the first metric comprises:
comparing the first metric to: (i) a first quartile that corresponds to a reference Parameter D value of a normal subject that is less than −5.077; (ii) a second quartile that corresponds to an average Parameter D value between −5.076 and −3.631; (iii) a third quartile that corresponds to an average Parameter D value between −3.630 and −2.209; and a fourth quartile that corresponds to an average Parameter D value equal to or greater than −2.209; and
determining a cumulative rate of survival for the subject based on the comparison, wherein subjects within the fourth quartile have a lower cumulative rate of survival as compared to subjects in the first-third quartile.

17. The computer-program product of claim 16, wherein the first measurement curve indicates a volume of air exhaled over a time period and the second measurement curve indicates a rate of flow over a volume of air exhaled.

18. The computer-program product of claim 15, wherein the actions further include determining the presence of the airflow obstruction is associated with chronic obstructive pulmonary disease (COPD); and generating and implementing a treatment plan for COPD for the subject based on the determined presence of the airflow obstruction and/or the determined cumulative rate of survival for the subject.

19. The computer-program product of claim 15, wherein the actions further include:
performing at least a second curve-fitting on the second measurement curve, wherein the performing the second curve-fitting on the second measurement curve comprises:
applying a second function with at least two or more linear segments to a portion of the second measurement curve; and
determining a second metric based on an intersection point for the at least two or more linear segments of the piece-wise function;
performing at least a third curve-fitting on the second measurement curve, wherein the performing the third curve-fitting on the second measurement curve further comprises:
applying a third function around a highest point of the second measurement curve using a least squares minimization; and
determining a third metric from the highest point of the second measurement curve and a point where the applied function deviates from the second measurement curve;
comparing the first metric, the second metric, and the third metric to a plurality of threshold values; and
determining the presence or absence of airflow obstruction for the subject based on the comparison of the first metric, the second metric, and the third metric to the plurality of threshold values.

20. The computer-program product of claim 15, wherein the one or more thresholds comprise a 90^{th} percentile threshold of −4.083 or a 75^{th} percentile threshold of −4.083, and the presence of airflow obstruction for the subject is determined when the first metric is less than the 90^{th} percentile threshold of −4.083 or the 75^{th} percentile threshold of −3.261.
